(19) United States
(12) Patent Application Publication
(10) Pub. No.: US 2013/0172752 A1
(43) Pub. Date: Jul. 4, 2013

(54) ULTRASOUND TRANSDUCER APPARATUS AND ULTRASOUND IMAGING SYSTEM AND IMAGING METHOD

(71) Applicant: Industrial Technology Research Institute, Hsinchu (TW)

(72) Inventors: Chang-Lin Hu, Kaohsiung City (TW); Shan-Yi Yang, Taichung City (TW); Shiow-Harn Lee, Hsinchu City (TW)

(73) Assignee: INDUSTRIAL TECHNOLOGY RESEARCH INSTITUTE, Hsinchu (TW)

(21) Appl. No.: 13/662,555

(22) Filed: Oct. 29, 2012

(30) Foreign Application Priority Data
Dec. 28, 2011 (TW) 100149285

(51) Int. Cl. A61B 8/14

(52) U.S. Cl. 600/447

(57) ABSTRACT

An ultrasound transducer apparatus includes an ultrasound transducer array consisting of multiple ultrasound transducer elements. The ultrasound transducer array is adapted for receiving and transmitting an ultrasound wave. A pulse controller is adapted for outputting multiple pulse signals to control the ultrasound transducer elements, respectively. By delaying output of the pulse signals, a plane wavefront of a transmitted ultrasound is controlled to have a different propagation direction. The pulse controller is able to activate part of the ultrasound transducer elements, corresponding to a region of interest for ultrasound wave receiving and transmitting.

### CROSS REFERENCE TO RELATED APPLICATIONS

This application claims the priority benefit of Taiwan application serial no. 100149285, filed on Dec. 28, 2011. The entirety of the above-mentioned patent application is hereby incorporated by reference herein and made a part of this specification.

### BACKGROUND

1. Technical Field

The disclosure relates to an ultrasound transducer apparatus and ultrasound imaging system and imaging method.

2. Related Art

Ultrasound imaging has been widely used, especially in the medical field, which can obtain the status of organs within a human body as an example.

Due to the advantages of easy access, low price, and high safety, medical ultrasound imaging devices have gradually had a higher popularity than other medical imaging technologies in recent years. However, the images produced by an ultrasound device must have sufficient quality to provide correct information for clinical diagnosis and analysis. Therefore, a high resolution ultrasound imaging technology is needed. One example of the high resolution ultrasound imaging technology is multipoint focusing transmission technology which, however, reduces the frame rate and hence affects the dynamic image scan rate. Therefore, it usually needs to balance between the image resolution and frame rate.

The synthetic aperture focusing technique (SAFT) was first proposed in the 1980s and has been considered as a method capable of effectively increasing the image resolution without reducing the frame rate too much. The SAFT can analyze received ultrasound channel data in a time-domain or a frequency-domain.

In recent years, the demand for new applications of diagnostic ultrasound, such as, 3D/4D imaging, cardiac imaging and ultrasound elastography have been increased dramatically. Therefore, if the high frame rate condition is satisfied, it can be applied in at least the foregoing ultrasound imaging applications, which can provide the physicians with more information and methods for clinical diagnosis.

### SUMMARY

An ultrasound transducer apparatus provided in the disclosure includes an ultrasonic transducer array and a pulse controller. The ultrasound transducer array includes a plurality of ultrasound transducer elements for receiving and transmitting an ultrasound wave. The pulse controller is adapted for outputting a plurality of pulse signals to control the ultrasound transducer elements, respectively. By delaying output of the pulse signals, a plane wavefront of a transmitted ultrasound is controlled to have a different propagation direction. The pulse controller is able to activate part of the ultrasound transducer elements corresponding to a region of interest for ultrasound wave receiving and transmitting.

An ultrasound imaging system, provided in the disclosure, includes an ultrasound transducer array, a pre-image process unit and a post-image process unit. The ultrasound transducer includes a plurality of ultrasound transducer elements. The ultrasound transducer array is able to activate part of the ultrasound transducer elements or selected part of the ultrasound transducer elements, according to an aperture needed by a region of interest. The ultrasound transducer array receives a plurality of reflected waves with different wavefront angles for transforming into a plurality of electrical signals. The pre-image process unit is adapted for receiving the electrical signals to process them into a plurality of corresponding digital sub-images. The post-image process unit is adapted for phase correcting and demodulating the digital sub-images and further compounding the digital sub-images into an ultrasound image.

An ultrasound imaging method is provided in the disclosure. The imaging method further includes performing a first ultrasound imaging. In the first ultrasound imaging, all the ultrasound transducer elements are activated and receiving an ultrasonic wave with a zero degree wavefront and a pair of reflected waves with non-zero degree wavefronts that are reflected back by a detection target. The non-zero degree wavefronts have symmetrical positive and negative wavefront angles. A pre-image processing is performed for receiving the electrical signals transmitted from the ultrasound transducer array and processing the electrical signals into a plurality of digital sub-images. A post-image processing is performed for phase correcting and demodulating the digital sub-images and further compounding the digital sub-images to obtain a first ultrasound image. The region of interest is determined from the first ultrasound image. The imaging method further includes performing a second ultrasound imaging. In the second ultrasound imaging, the part of the ultrasound transducer elements are activated or the part of the activated ultrasound transducer elements are selected according to the aperture needed by the region of interest. The receiving of the ultrasound wave with a zero degree wavefront and the pair of reflected waves with non-zero degree wavefronts that are reflected back by the detection target are repeated. The non-zero degree wavefronts have symmetrical positive and negative wavefront angles. The pre-image processing is repeated. The post-image processing is repeated to obtain a second ultrasound image with respect to the region of interest.

The imaging method further includes performing a correlation calculation between the first ultrasound image and the second ultrasound image with respect to the region of interest to obtain a third ultrasound image, and further performing a correlation calculation between the second ultrasound image and the third ultrasound image to obtain a fourth ultrasound image.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic view illustrating an ultrasound transmitting mechanism (zero degree wavefront) according to an exemplary embodiment.

FIG. 2 is a schematic view illustrating an ultrasound transmitting mechanism (non-zero degree wavefront) according to an exemplary embodiment.

FIG. 3 is a schematic view illustrating an ultrasound transducer apparatus according to an exemplary embodiment.

FIG. 4 is a schematic view illustrating the control of the ultrasound transmitting aperture with respect to a ROI according to an exemplary embodiment.

FIG. 5 is a functional block diagram illustrating an ultrasound imaging system according to an exemplary embodiment.

FIG. 6A to FIG. 6C are schematic views illustrating sub-images obtained for ultrasound waves at different wavefront angles according to an exemplary embodiment.

FIG. 7 is a schematic view illustrating a simulation of compounding of different numbers of sub-images according to an exemplary embodiment.

FIG. 8 is a schematic view illustrating a mechanism of correlation calculation with respect to a ROI according to an exemplary embodiment.

FIG. 9 is a schematic view illustrating the mechanism and result of cross correlation calculation according to an exemplary embodiment.

FIG. 10 is a schematic view illustrating an ultrasound imaging system according to an exemplary embodiment.

### DETAILED DESCRIPTION OF DISCLOSED EMBODIMENTS

In the high speed ultrasound imaging mechanism of this disclosure, a plurality of ultrasound transducer element is used to form an ultrasound transducer apparatus with a single array or multiple arrays, serving as an ultrasound probe for transmitting a plane wave. The probe has multiple channels formed from the ultrasound transducer elements for transmitting a plane wave. The aperture size of the ultrasound probe can be adjusted by controlling the number of the channels activated. In addition, by controlling output time sequence of the channels, the ultrasound probe can be controlled to have different incident angles for ultrasound transmission. After backscatter signals have been received, a series of subsequent processing can be performed on the received backscatter signals in a frequency-domain.

The aperture adjustment of the ultrasound probe can be done by directly controlling the range of activated channels of the array corresponding to a region of interest (ROI), but also be achieved by selectively processing the channel signals corresponding to the ROI. That is, for example, all the channels of the ultrasound transducer apparatus can transmit and receive ultrasound waves but subsequent processing is performed with respect to the selected ROI channels to reduce image processing time. The signals with different incident angles are received and processed to obtain a plurality of sub-images. These sub-images are compounded to reconstruct a new image.

Ultrasound imaging in this manner can reduce the acoustic output power for purpose of improving safety and electrical power saving. In addition, selectively processing the channel signals can significantly reduce the signal processing time and increase the imaging frame rate. Moreover, the image compounding technology is capable of effectively reducing speckle noises and increasing lateral resolution.

Below, the ultrasound imaging system and method are described by way of exemplary embodiments. However, this disclosure is not intended to be limited to these exemplary embodiments.

To achieve high speed imaging, one way as an example is using a transmission manner in which the ultrasound transmitting source is caused to generate a plane wave signal. In this transmission manner, only one time of ultrasound signal transmission is needed for obtaining an image after the probe receives the backscatter signal, which can effectively reduce the waiting time for ultrasound transmitting and receiving, such that the frame rate is greatly increased. However, in this transmission manner, the ultrasound signals have poor focusing capability and intensity, causing the magnitude of the ultrasound backscatter signals to be relatively small, such that the signal-to-noise ratio (SNR), image contrast and spatial resolution at the receiving end are inferior to that of images obtained in the conventional ultrasound focusing transmission manner.

The method proposed in this disclosure is to control the number of ultrasound transducer elements activated in the ultrasound array probe to achieve an aperture adjustment for plane wave transmission. By use of time delay of transmission of each ultrasound transducer element of the ultrasound array, the ultrasound probe can be controlled to generate different incident angles for plane wave transmission.

In this disclosure, when the ultrasound array probe transmits ultrasound energy, not all channels are required to transmit an ultrasound signal. The signal transmitting and receiving of each channel corresponds to one probe element, and the corresponding relationship between the channels and elements can be controlled through a multiplexer (MUX). Channel selection in this manner can achieve the aperture adjustment, and the ultrasound imaging in this aperture adjustment manner can reduce the ultrasound transmission energy, thereby achieving the improved safety and the electrical power saving. During ultrasound imaging, motion artifact may occur if a relative movement occurs between the probe and a tissue organ. In this disclosure, it is not necessary to process the channel signals received by all elements. Rather, the channel signals corresponding to the ROI are selectively processed, which can significantly reduce the signal processing time and increase the imaging frame rate. Therefore, this disclosed ultrasound imaging system and method can effectively reduce the occurrence of motion artifact and can thus be applied in the cardiac sonography and vascular imaging applications. In this disclosure, a series of processing is performed to the channel signals generated by receiving the backscatter signals in a frequency-domain. This avoids the conventional manner in which a delay and sum processing is performed in a time-domain, which can effectively increase the calculation speed. Further, the use of image compounding technology can effectively reduce the occurrence of speckle noises, thus increasing the lateral resolution of the image.

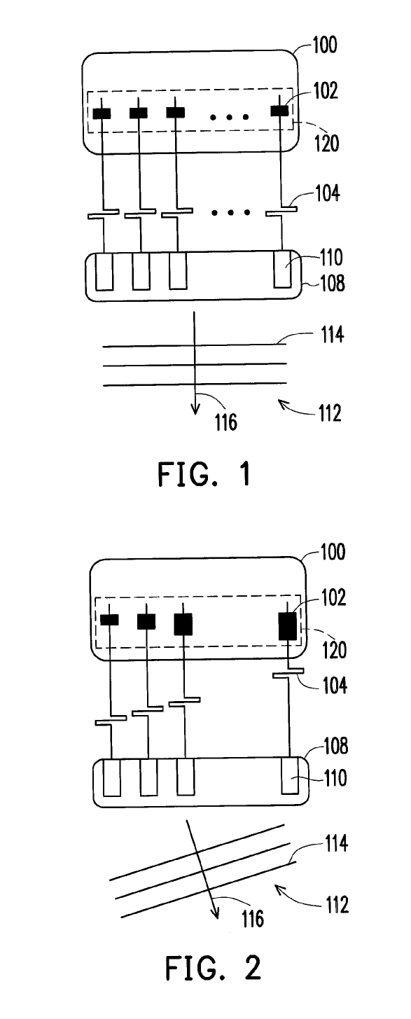

FIG. 1 is a view illustrating an ultrasound transmitting mechanism (zero degree wavefront) according to an exemplary embodiment. In this embodiment, the ultrasound wave is a plane wave. Referring to FIG. 1, the transmitting of the ultrasound wave 112 may be controlled by a pulse controller 120 of a transmitting module 100. The pulse controller 120 outputs a plurality of pulse signals with a delay setting. When pulse signals of all channels are controlled to have a same time delay 102, same pulse signals 104 can be synchronously generated in the respective channels to thereby form the plane wave. As such, a propagation direction 116 of a wavefront 114 of the ultrasound wave 112 is perpendicular to a plane of an ultrasound transducer array 108.

Each pulse signal 104 excites a corresponding ultrasound transducer element 110 of the ultrasound transducer array 108, each ultrasound transducer element 110 can be used to transmit or receive an ultrasound wave 112, and the propagation direction 116 of the transmitted ultrasound is shown in FIG. 1. It is assumed that the angle perpendicular to the ultrasound transducer array 108 is defined as zero degree. The ultrasound transducer elements 110 as an example are made from a piezoelectric material and are excited to vibrate to generate the ultrasound wave 112 according to the electrical pulse signal 104. On the contrary, when receiving a reflected ultrasound wave, the ultrasound transducer elements 110 also generates an electrical signal to be received by a receiving module 150 (not shown in FIG. 1) for subsequent processing to form an image.

FIG. 2 is a view illustrating an ultrasound transmitting mechanism (non-zero degree wavefront) according to an exemplary embodiment. Referring to FIG. 2, the transmitting mechanism is first discussed. If it is needed to change the angle of the wavefront 114 of a transmitted ultrasound wave 112, the pulse controller 120 of the transmitting module 100 may control the time delay between the pulse signals 104. Specifically, by adjusting a transmitting delay amount 102 of the pulse signal of each channel such that the transmitting delay amount 102 of each channel increases sequentially, the pulse signal 104 of each channel can be controlled to sequentially excite the ultrasound transducer elements 110 and, as such, the propagation direction 116 of the wavefront 114 of the ultrasound wave 112 is changed by the delay setting, which is no longer perpendicular to the array plane of the ultrasound transducer array 108.

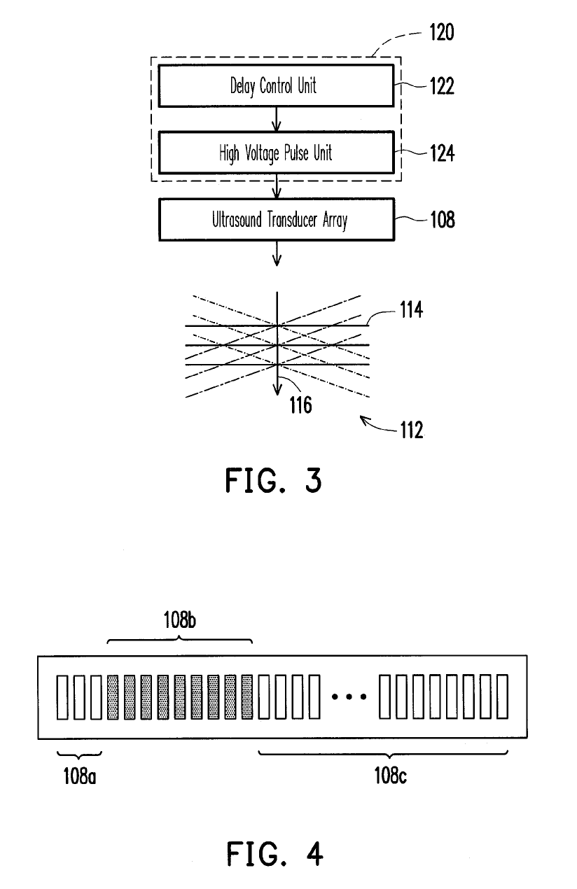

Based on the above operation mechanism, in practical operation, an odd number by at least three, of ultrasound wave 112 are transmitted for each imaging, which include the zero degree ultrasound wave 112 of FIG. 1 as well as two ultrasound waves 112 with symmetrical positive and negative angles according to the mechanism of FIG. 2. FIG. 3 is a view illustrating the ultrasound transducer apparatus according to one exemplary embodiment.

Referring to FIG. 3 as well as the corresponding part of FIG. 1, the ultrasound transducer apparatus includes a pulse control unit 120 and an ultrasound transducer array 108. The ultrasound transducer array 108 consists of the plurality of ultrasound transducer elements 110 which form a plane array. The pulse controller 120 outputs a plurality of pulse signals 104 controlling the ultrasound transducer elements 110, respectively. By delaying output of the pulse signals 104, the plane wavefronts 114 of the transmitted ultrasound waves 112 can be controlled to have different propagation directions 116.

As such, the pulse controller 120 can activate a part of the ultrasound transducer elements 110 corresponding to a region of interest (ROI). The part of the ultrasound transducer elements 110 transmit and receive the ultrasound waves with respect to the ROI.

The pulse controller 120 may include a delay control unit 122 and a high voltage pulse unit 124. The time for the high voltage pulse unit 124 to generate the pulse signal 104 is adjusted by the time delay of the delay control unit 122. The ultrasound transducer array 108 receives the pulse signal 104 generated by the high voltage pulse unit 124 so as to generate the ultrasound wave 112. A plane wavefront 114 of which is shown in FIG. 3. In the embodiment, the propagation direction 116 of the wavefront 114 in solid line is at a zero degree. The propagation directions of the other two wavefronts have positive/negative deviations, such as, 1° and -1° deviations, from the zero degree direction, respectively, such that two ultrasound waves 112 with symmetrical angles are transmitted. The two ultrasound waves 112 with the symmetrical wavefront angles facilitate the cancellation of an image sidelobe, which will be discussed later.

FIG. 4 is a view illustrating the control of the ultrasound transmitting aperture with respect to the ROI. Referring to FIG. 4, the ultrasound transducer array 108 includes the plurality of ultrasound transducer elements 110. According to an aperture needed by the ROI 108b, it is possible to activate part of the ultrasound transducer elements 110 or select part of the activated ultrasound transducer elements 110. It is noted, however, that the disclosed ultrasound imaging system and method are not limited to any particular implementations described herein. In this embodiment, taking one part of ultrasound transducer elements 110 of the ultrasound transducer array 108 for illustration, the ultrasound transducer elements 110 that correspond to the ROI 108b are activated, while the other ultrasound transducer elements 110 that correspond to regions of non-interest (ROM) 108a and 108c are not activated. Therefore, the scope of ultrasound imaging is within the ROI 108b. That is to say, in the embodiment, the ultrasound transmitting aperture is just corresponding to the ROI 108b.

In this disclosure, part of the ultrasound transducer elements that correspond to the ROI are activated, which can at least reduce the processing time needed for imaging, reduce the acoustic output power and save the electrical power.

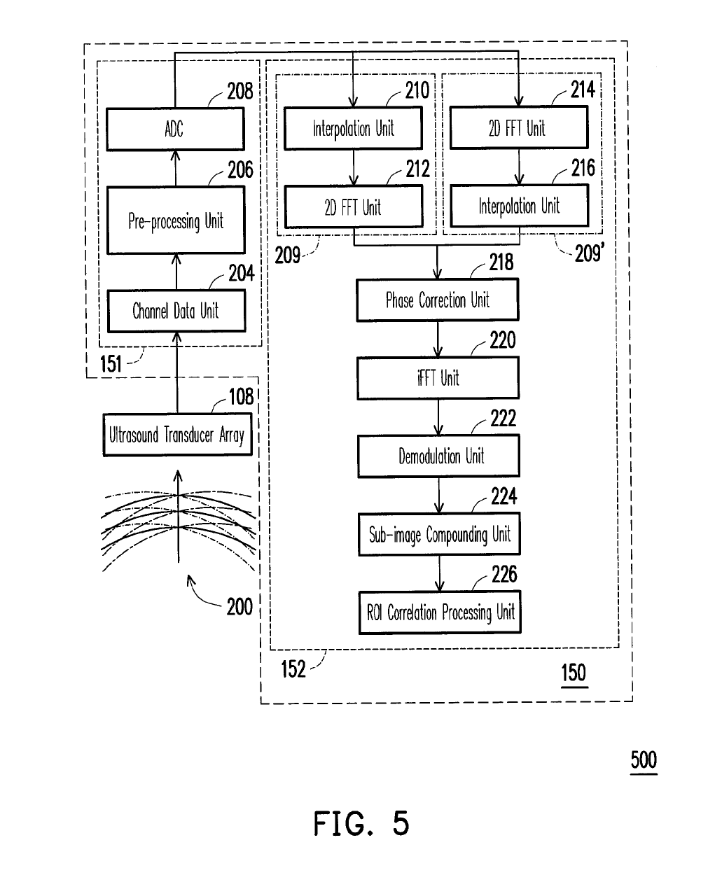

A receiving mechanism is described below. FIG. 5 is a functional block diagram of an ultrasound imaging system according to one exemplary embodiment. Referring to FIG. 5, the ultrasound imaging system 500 also includes a receiving module 150 and an ultrasound transducer array 108. When the transmitting module 100 controls the ultrasound transducer array 108 to transmit a plurality of plane ultrasound waves 112, these ultrasound waves 112 are reflected by a target, such as, a body tissue, to form reflected waves 200 returning to the ultrasound transducer array 108 and received by the receiving module 150. Based on the characteristics of reflection, the wavefronts of the reflected waves 200 that are reflected back do not maintain to be a plane. Instead, they have different wavefront angles. However, they still correspond to the odd number of transmitted ultrasound waves 112, i.e. the number of the reflected waves is also an odd number, including a reflected wave with a zero degree wavefront and at least a pair of reflected waves with non-zero degree wavefronts that have symmetrical positive and negative wavefront angles. Specifically, upon receiving the reflected waves with different wavefront angles, the ultrasound transducer array 108 transforms the reflected waves 200 into a plurality of electrical signals and transmits these electrical signals to the receiving module 150.

The receiving module 150 includes a pre-image processing unit 151 and a post-image processing unit 152. The pre-image processing unit 151 performs an image pre-processing, i.e. receiving the electrical signals transmitted from the ultrasound transducer array 108 to process them into a plurality of corresponding digital sub-images. The post-image processing unit 152 performs a post-imaging processing, i.e. phase correcting and demodulating the digital sub-images and further compounding these sub-images into an ultrasound image. In addition, the ultrasound imaging system 500 further includes the pulse controller 120 as shown in FIG. 3, operation and function of which are the same as those described above and explanation thereof is therefore not repeated herein.

Specifically, the pre-image processing unit 151 includes a channel data unit 204, a pre-processing unit 206, and an analog-to-digital converter (ADC) 208. The channel data unit 204 receives the electrical signals transmitted and transformed by the ultrasound transducer array 108 corresponding to the reflected waves 200. The pre-processing unit 206 then pre-processes the received electrical signals, including amplifying and filtering noises from the electrical signals to obtain a plurality of groups of pre-amplified signals. The ADC 208 then receives and converts these pre-amplified signals into a plurality of digital sub-images.

The post-image processing unit 152 includes an interpolation and fast Fourier transform (FFT) unit 209, 209′, a phase correction unit 218, an inverse FFT (iFFT) unit 220, a demodulation unit 222, an image compounding unit 224 and a ROI correlation processing unit 226.

In the imaging mechanism adopted by this disclosure, a time-domain plane image is achieved after a fast phase correction is performed in the frequency-domain. Therefore, a FFT is required to be performed to the digital sub-images. However, there is sampling insufficiency problem in converting the analog reflected wave signals into digital signals and, therefore, up-sampling is needed.

Therefore, the interpolation and FFT unit performs the interpolation and fast Fourier transform, i.e. performing sampling-interpolation and fast Fourier transform to the digital sub-images respectively to generate a plurality of frequency-domain digital sub-images. The interpolation and FFT unit 209 includes an interpolation unit 210 and a two-dimensional fast Fourier transform (2D FFT) unit 212. The interpolation and FFT unit 209′ operating in another manner includes a 2D FFT unit 214 and an interpolation unit 216. The post-image processing unit 152 may, for example, utilize the interpolation and FFT unit 209, such that the interpolation unit 210 first performs sampling-interpolation for increasing sampling points (up-sampling) in the time-domain, and the 2D FFT unit 212 then performs a time-domain to frequency-domain transform to an interpolated data matrix to obtain a plurality of frequency-domain sub-images.

Another manner is to utilize the interpolation and FFT unit 209′, such that the 2D FFT unit 214 first performs the time-domain to frequency-domain transform to the digital sub-images to obtain a plurality of frequency-domain sub-images and the interpolation unit 216 then performs the sampling-interpolation for increasing sampling points by up-sampling to have multiple frequency-domain sub-images.

After the time-domain to frequency-domain transformation, the above-mentioned frequency-domain digital sub-images are received by the phase correction unit 218 to be corrected in phase. The frequency-domain digital sub-images with phase correction are then transformed into a plurality of time-domain sub-images by the iFFT unit 220.

The actual image data is carried on an ultrasound baseband data. To achieve an image with varied brightness, the time-domain images are required to be demodulated using the demodulation unit 222 and baseband data is required to be removed. As such, the sub-images corresponding to the reflected waves of various wavefront angles can be achieved.

It is understandable that each sub-image corresponds to a reflected wave with a wavefront angle and is generated through the above-mentioned post-image processing unit. That is, the sub-images include the sub-image corresponding to the zero degree wavefront, and at least also include the two sub-images corresponding to the wavefronts that have the same deviation angles in the positive and negative directions. There may be more reflected wave information corresponding to more wavefront angles depending upon actual requirements. The symmetry in the wavefront angles facilitates the cancellation of image noises, which will be described later.

After the plurality of sub-images is obtained, the sub-image compounding unit 224 compounds the sub-images into an ultrasound image.

Here, compounding more sub-images corresponding to more different wavefront angles would have better results but it would take relatively more time. Therefore, a suitable number of sub-images can be selected for compounding. The image noises remaining in each sub-image, such as, sidelobes, can be removed by the compounding of the sub-image compounding unit 224.

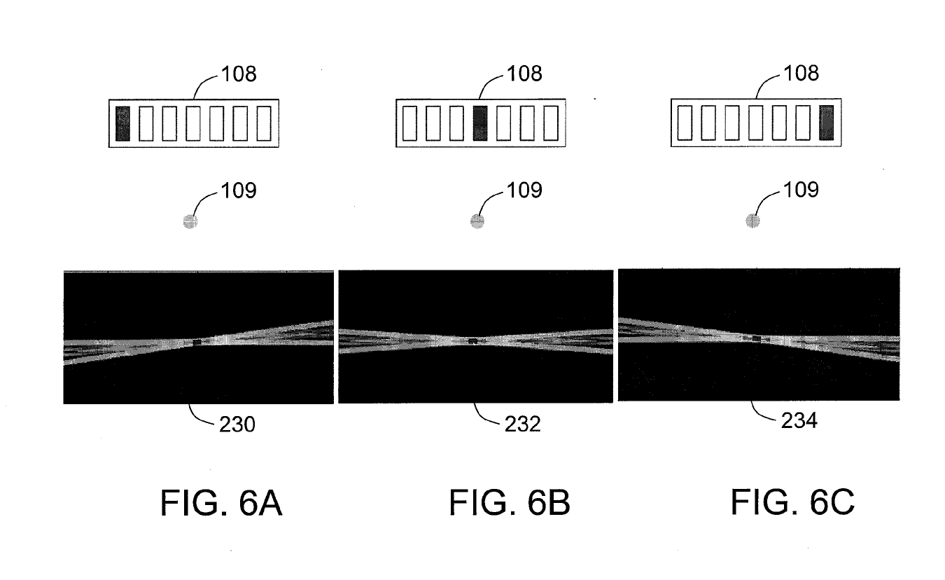

The mechanism of cancellation of sub-image noises is first discussed below. FIG. 6A to FIG. 6C are views illustrating sub-images obtained for ultrasound waves at different wavefront angles. Referring to FIG. 6A, the ultrasound transducer array 108 images a detection target 109 at a “−10° ” wavefront angle, and the intensity distribution of the obtained sub-image 230 is shown in the lower figure of FIG. 6A. The horizontal axis of the lower figure of FIG. 6A represents a lateral position on the ultrasound transducer array 108 with respect to the detection target 109, the vertical axis represents a distance to the ultrasound transducer array 108. The central bright point illustrates the brightness intensity of the detection target 109 at its actual position, i.e., the central bright point is the mainlobe of the image. However, due to the characteristics of plane wave reflection, there are image sidelobes at other positions.

Likewise, FIG. 6B illustrates a sub-image 232 of the detection target 109 when the ultrasound wave is transmitted with a zero degree wavefront. A local image mainlobe still appears at the actual position of the detection target 109, while an extension direction of the image sidelobe is slightly different. FIG. 6C illustrates a sub-image 234 of the detection target 109 when the ultrasound wave is transmitted with a 10° wavefront. The scatter points at the actual position of the detection target still form a local image mainlobe, while the extension direction of the image sidelobe is substantially symmetrical with the extension direction of the image sidelobes of FIG. 6A. If these sub-images are compounded, the image mainlobe is intensified, while the image sidelobe is weakened. After a threshold filtering, the image mainlobe becomes the content of the image.

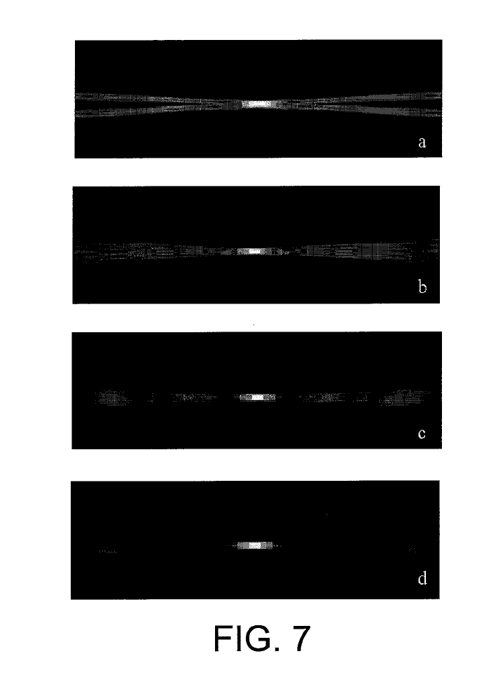

FIG. 7 is a view illustrating a simulation of compounding of different numbers of sub-images. Referring to FIG. 7, in FIG. 7(a), the ultrasound waves include an ultrasound wave with a zero degree wavefront as well as two ultrasound waves with 10° and −10° wavefronts. The compounding result of the three sub-images is the same as the result of compounding the sub-images 230, 232 and 234 of FIG. 6A, FIG. 6B and

FIG. 6C, in which the sidelobe still exists. FIG. 7(b) to FIG. 7(d) illustrate the sub-image compounding results when additional sub-images corresponding to ultrasound waves with wavefronts of other angles are added. As can be seen from the simulated image compounding results, the image sidelobe can be more easily cancelled when more sub-images corresponding to more ultrasound waves with different wavefront angles are compounded. However, more ultrasound waves transmitted at different wavefront angles require increased time for image processing. Therefore, in the embodiment, compounding, for example, five sub-images is typically sufficient, and the image sidelobe can be cancelled using other correlation calculation mechanism.

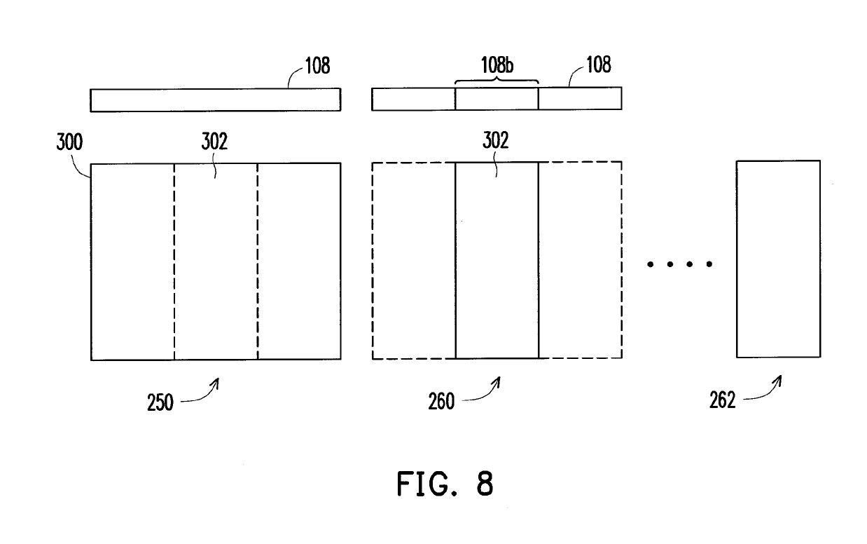

This disclosure proposes to perform a correlation calculation to the ROI, which is carried out by the ROI correlation processing unit 226 of the ultrasound imaging system 500 of FIG. 5. FIG. 8 is a view illustrating a mechanism of correlation calculation with respect to the ROI. Referring to FIG. 5 and FIG. 8, in the first imaging, the ultrasound image obtained by the ultrasound transducer array 108 at the first time is a first ultrasound image 250. At this time, the ROI has not been chosen yet and, therefore, the ultrasound transducer elements of the ultrasound transducer array 108 are all activated. Later, the ROI 302 is determined from the first ultrasound image 250, and the other portions of the image belong to the RONI 300. After the ROI 302 has been determined, the ultrasound transducer array 108 is controlled in the manner of FIG. 4 to activate a region 108b of the ultrasound transducer array 108 that corresponds to the ROI 302 according to an aperture needed by the ROI 302. The above-described pre-image processing and post-image processing are repeated to obtain a second ultrasound image 260 with respect to the ROI 302.

A correlation, for example, a cross correlation, is calculated between the first ultrasound image 250 and the second ultrasound image 260 with respect to the data of the ROI. This cross correlation is used as a weighted value for adjusting the first ultrasound image 250 and the second ultrasound image 260 to obtain a third ultrasound image with higher quality, such as, with increased resolution, contrast, signal to noise ratio, or the like. The correlation calculation can be repeatedly conducted with respect to the same image region. For example, after obtaining the third ultrasound image by calculating the cross correlation between the first ultrasound image 250 and the second ultrasound image 260, a correlation calculation such as the cross correlation calculation can be performed between the second ultrasound image 260 and the third ultrasound image to obtain a fourth ultrasound image 262. More ultrasound images may be obtained in this manner by analogy.

However, the cross correlation calculation is not limited to the manner as described above. Rather, the cross correlation calculation can be performed between two continuous ultrasound images. For example, having obtained the second ultrasound image, a fifth ultrasound image is continuously and directly measured, and the correlation calculation is performed between the continuously measured second ultrasound image and fifth ultrasound image to obtain a sixth ultrasound image with good quality. However, this depends on the requirements of time to be consumed and the lateral resolution.

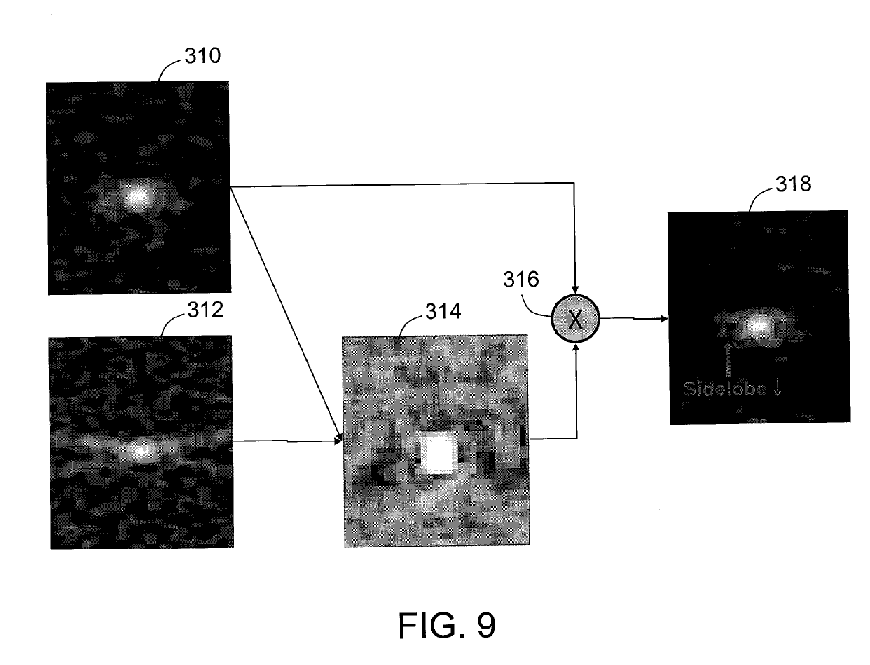

FIG. 9 is a view illustrating the mechanism and result of cross correlation calculation according to one exemplary embodiment. Referring to FIG. 9, taking a point spread function as an example, the simulated object at the same point is scanned by ultrasound waves in different manners. The resulted ultrasound image 310 is an original point spread function, which is an ideal point spread function with a narrow mainlobe and a low sidelobe. The point spread function of the ultrasound image 312 is a non-ideal point spread function with a wide mainlobe and a high sidelobe. By performing a cross correlation calculation between the point spread functions of the two ultrasound images, a correlation map 314 can be obtained which has a large weighted value at the mainlobe position and a small weighted value at the sidelobe position.

The point spread function of the ultrasound image 312 is multiplied by the correlation map 314 using a multiplier 316. The mainlobe in an ultrasound image 318 of the weighted point spread function becomes narrower and the sidelobe becomes lower. As such, the image quality is improved.

In other words, the image compounding unit 224 of FIG. 5 does not need to compound too many sub-images. The remaining image noises can be otherwise cancelled in a simpler manner by the processing of the ROI correlation processing unit 226.

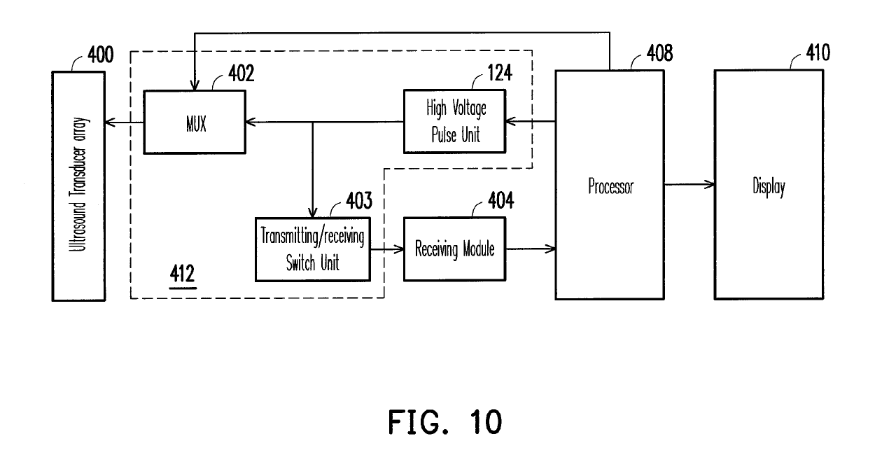

FIG. 10 is a view illustrating an ultrasound imaging system according to one exemplary embodiment. Referring to FIG. 10, the ultrasound imaging system includes an ultrasound transducer array 400, a transmitting module 412, a receiving module 404, a processor 408, and a display 410. The transmitting module 412 includes a multiplexer 402, a transmitting/receiving switch unit 403 and a high voltage pulse unit 124. The function of the transmitting module 412 is the same as the function of the transmitting module 100 of FIG. 1. The function of the receiving module 404 is the same as the function of the receiving module 150 of FIG. 5. The high voltage pulse unit 124 and the receiving module 404 are coupled to the ultrasound transducer array 400 through the switching of the multiplexer 402 to realize the ultrasound wave transmitting and receiving operations. In addition, when the high voltage pulse unit 124 transmits a pulse signal 104, the transmitting/receiving switch unit 403 functions like a high impedance component to block the high voltage pulse signal 104 from entering the receiving module 404 thus protecting various module elements. However, after the high voltage pulse signal 104 has been transmitted, the transmitting/receiving switch unit 403 functions like a low impedance component to permit the receiving module 404 to receive a backscatter signal from the ultrasound transducer array 400. After the receiving module 404 obtains the ultrasound backscatter signals of all channels, the processor 408 performs a subsequent process series of signal and data processing on the received signals. The processed signals may be displayed on the display 410.

While the processor 408 is illustrated as a separate unit in the above description, control and calculation in the transmitting module 412, receiving module 404 and display 410 may be integrated into the processor 408 in practice.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An ultrasound transducer apparatus comprising:
an ultrasound transducer array comprising a plurality of ultrasound transducer elements, the ultrasound transducer array for receiving and transmitting an ultrasound wave;
a pulse controller, outputting a plurality of pulse signals to control the ultrasound transducer elements, respectively, wherein the pulse signals are controlled to output by a delay setting, and a plane wavefront of the transmitted ultrasound wave is controlled according to the delay setting to have a different propagation direction,
wherein the pulse controller is able to activate a part of the ultrasound transducer array corresponding to a region of interest for ultrasound wave transmitting and receiving.

2. An ultrasound imaging system comprising:
an ultrasound transducer array comprising a plurality of ultrasound transducer elements, wherein the ultrasound transducer elements of the ultrasound transducer array can be activated a part of the ultrasound transducer elements or a part of the ultrasound transducer elements activated is selected, according to an aperture needed by a region of interest, and the ultrasound transducer array receives a plurality of reflected waves with different wavefront angles for transforming into a plurality of electrical signals;
a pre-image processing unit, to receive and process the electrical signals to process into a plurality of corresponding digital sub-images; and
a post-image processing unit, for correcting phase and demodulating the digital sub-images and further compounding the digital sub-images into an ultrasound image.

3. The ultrasound imaging system according to claim 2, further comprising a pulse controller adapted for outputting a plurality of pulse signals to control the ultrasound transducer elements, respectively, wherein, an ultrasound wave is transmitted according to a delay setting with the pulse signals such that a plane wavefront of the ultrasound wave is controlled to have a different propagation direction,
wherein the pulse controller is able to activate the part of the ultrasound transducer elements corresponding to the region of interest, and the part of the ultrasound transducer elements receive and transmit ultrasound waves with respect to the region of interest.

4. The ultrasound imaging system according to claim 2, wherein the pre-image processing unit comprises:
a channel data unit receiving the electrical signals transformed by the ultrasound transducer array corresponding to the reflected waves;
a pre-processing unit amplifying and filtering noises from the electrical signals to obtain a plurality of groups of pre-amplified signals; and
an analog to digital converter to receive and convert the groups of pre-amplified signals into the digital sub-images.

5. The ultrasound imaging system according to claim 2, wherein the post-image processing unit comprises:
an interpolation and fast Fourier transform unit performing sampling-interpolation to the digital sub-images and fast Fourier transforming the digital sub-images into a plurality of frequency-domain digital sub-images;
a phase correction unit receiving the frequency-domain digital sub-images for phase correction;
an inverse fast Fourier transform unit transforming the corrected frequency-domain digital sub-images into a plurality of images in a time-domain;
a demodulation unit demodulating the time-domain images and removing baseband data to obtain a plurality of sub-images; and
a sub-image compounding unit compounding the sub-images into an ultrasound image.

6. The ultrasound imaging system according to claim 5, wherein the interpolation and fast Fourier transform unit comprises:
an interpolation unit performing sampling interpolation for up-sampling in a time-domain; and
a two dimensional fast Fourier transform unit performing a time-domain to frequency-domain transformation to a data matrix generated by the interpolation unit to obtain the frequency-domain sub-images.

7. The ultrasound imaging system according to claim 5, wherein the interpolation and fast Fourier transform unit comprises:
a two dimensional fast Fourier transform unit performing a time-domain to frequency-domain transformation to the digital sub-images to obtain the frequency-domain sub-images; and
an interpolation unit performing sampling interpolation for up-sampling the frequency-domain sub-images to increase sampling points.

8. The ultrasound imaging system according to claim 2, wherein the number of the reflected waves is an odd number comprising a reflected wave with a zero degree wavefront and at least a pair of reflected waves with symmetrical positive and negative wavefront angles.

9. The ultrasound imaging system according to claim 2, further comprising a region of interest correlation processing unit, wherein the ultrasound image obtained by the ultrasound transducer array at the first time is a first ultrasound image, the ultrasound transducer elements are all activated, and the region of interest is determined from the first ultrasound image to control the ultrasound transducer array to activate the part of the ultrasound transducer elements or select the part of the ultrasound transducer elements activated, according to the aperture needed by the region of interest to obtain a second ultrasound image,
wherein a correlation calculation is performed between the first ultrasound image and the second ultrasound image with respect to the region of interest to obtain a third ultrasound image, and a further correlation calculation is performed between the second ultrasound image and the third ultrasound image to obtain a fourth ultrasound image.

10. The ultrasound imaging system according to claim 2, further comprising a region of interest correlation processing unit, wherein the ultrasound image obtained by the ultrasound transducer array at the first time is a first ultrasound image, the ultrasound transducer elements are all activated, and the region of interest is determined from the first ultrasound image to control the ultrasound transducer array to activate the part of the ultrasound transducer elements or select the part of the ultrasound transducer elements activated, according to the aperture needed by the region of interest to obtain a second ultrasound image,
wherein a correlation calculation is performed between a directly continuously measured fifth ultrasound image and the second ultrasound image to obtain a sixth ultrasound image.

11. An ultrasound imaging method comprising:
providing an ultrasound transducer array, wherein the ultrasound transducer array comprises a plurality of ultrasound transducer elements, the ultrasound transducer array is able to activate part of the ultrasound transducer elements or select part of the ultrasound transducer elements activated, according to an aperture needed by a region of interest, and the ultrasound transducer array receives a plurality of reflected waves with different wavefront angles for transforming into a plurality of electrical signals;
performing a first ultrasound imaging, comprising:
activating all the ultrasound transducer elements and receiving a reflected wave with a zero degree wavefront and a pair of reflected waves with non-zero degree wavefronts that are reflected back by a detection target, the non-zero degree wavefronts having symmetrical positive and negative wavefront angles;
performing a pre-image processing for receiving the electrical signals transmitted from the ultrasound transducer array and processing the electrical signals into a plurality of digital sub-images; and
performing a post-image processing for phase correcting and demodulating the digital sub-images and further compounding the digital sub-images to obtain a first ultrasound image, wherein the region of interest is determined from the first ultrasound image;
performing a second ultrasound imaging, comprising:
activating the part of the ultrasound transducer elements or select the part of the ultrasound transducer elements activated according to the aperture needed by the region of interest, repeating the receiving of the reflected wave with a zero degree wavefront and the pair of reflected waves with non-zero degree wavefronts that are reflected back by the detection target, the non-zero degree wavefronts having symmetrical positive and negative wavefront angles;
repeating the pre-image processing; and
repeating the post-image processing to obtain a second ultrasound image with respect to the region of interest; and
performing a correlation calculation between the first ultrasound image and the second ultrasound image with respect to the region of interest to obtain a third ultrasound image, and further performing a correlation calculation between the second ultrasound image and the third ultrasound image to obtain a fourth ultrasound image.

12. The ultrasound imaging method according to claim 11, wherein the pre-image processing comprises:
receiving the electrical signals transformed by the ultrasound transducer array corresponding to the reflected waves;
amplifying and filtering noises from the electrical signals to obtain a plurality of groups of pre-amplified signals; and
receiving the groups of pre-amplified signals to convert into the digital sub-images.

13. The ultrasound imaging system according to claim 11, wherein the post-image processing comprises:
performing an interpolation and fast Fourier transform for sampling-interpolation to the digital sub-images and fast Fourier transforming the digital sub-images into a plurality of frequency-domain digital sub-images;
phase correcting the frequency-domain digital sub-images;
transforming the corrected frequency-domain digital sub-images into a plurality of images in a time-domain;
demodulating the time-domain images and removing baseband data to obtain a plurality of sub-images; and
compounding the sub-images into the first ultrasound image or the second ultrasound image.

14. The ultrasound imaging system according to claim 13, wherein the interpolation and fast Fourier transform comprises:
performing sampling interpolation for up-sampling in a time-domain; and
performing a time-domain to frequency-domain transformation on a data matrix generated by the interpolation unit to obtain the frequency-domain sub-images.

15. The ultrasound imaging system according to claim 13, wherein the interpolation and fast Fourier transform comprises:
performing a time-domain to frequency-domain transform to the digital sub-images to obtain the frequency-domain sub-images; and
performing sampling interpolation for up-sampling the frequency-domain sub-images.
